(19) United States
(12) Patent Application Publication
(10) Pub. No.: US 2005/0085451 A1
(43) Pub. Date: Apr. 21, 2005

(54) Compound and method for enhancing the cholesterol lowering property of plant sterol and stanol esters

(76) Inventor: Timothy P. Carr, Lincoln, NE (US)

Correspondence Address: SUITER WEST PC LLO, 14301 FNB PARKWAY, SUITE 220, OMAHA, NE, 68154, US

(21) Appl. No.: 10/934,346

(22) Filed: Sep. 3, 2004

Related U.S. Application Data
(60) Provisional application No. 60/500,784, filed on Sep. 5, 2003.

(51) Int. Cl. A61K 31/56; A61K 31/225; A61K 31/202

(52) U.S. Cl. 514/171; 514/548; 514/560

(57) ABSTRACT

The present invention includes a compound which combines, through esterification, the natural cholesterol lowering properties of plant sterols or stanols with the natural cholesterol lowering properties of fatty acids, more particularly a stearic acid. This combination of substances provides a synergistic lowering of cholesterol by interfering with the normal absorption of cholesterol within the gastrointestinal tract of the digestive system and may assist in avoiding unwanted side effects which are typically experienced with commonly used cholesterol lowering drugs.

### CROSS REFERENCE TO RELATED APPLICATION

The present application claims priority under 35 U.S.C. §119(e) to the U.S. Provisional Application Ser. No. 60/500,784, filed on Sep. 5, 2003, which is herein incorporated by reference in its entirety.

### FIELD OF THE INVENTION

The present invention generally relates to compounds for use as dietary supplements that help lower serum cholesterol in humans. More particularly, the present invention relates to compounds created by from the combination, through processes such as esterification, of plant sterols or stanols with specific cholesterol-lowering fatty acids, such as stearic acid.

### BACKGROUND OF THE INVENTION

Elevated serum cholesterol, low-density lipoprotein (LDL) cholesterol, is a primary risk factor for atherosclerotic diseases, including coronary heart disease and stroke, See American Heart Association (2000), AHA dietary guidelines, Revision 2000A statement for healthcare professionals from the Nutrition Committee of the American Heart Association, Circulation 102: 2296-2311, which is herein incorporated by reference in its entirety. Reducing serum cholesterol concentration significantly reduces the risk of these diseases. Consequently, current dietary and drug therapies are designed to lower serum LDL cholesterol levels. Scientific evidence clearly indicates that lowering serum cholesterol levels will reduce the risk of heart disease and stroke, See National Institutes of Health (1985), Lowering blood cholesterol to prevent heart disease, NIH Consensus Development Conference statement. Arteriosclerosis 5: 404-412, which is herein incorporated by reference in its entirety. Therapy with cholesterol lowering drugs may prove effective. However, the use of these drugs has been associated with serious side effects in some people. A more desirable approach is to lower serum cholesterol through dietary means, such as reducing the amount of saturated fat or increasing dietary fiber. These dietary practices can be effective in some people, but for the majority of the population with marginal cholesterol levels their effects are limited. Finding alternative dietary strategies for the entire population is clearly desirable.

The cholesterol-lowering ability of plant sterols has been known for many years. Plant sterols and stanols, hereinafter referred to as plant sterols (stanols), lower blood cholesterol levels by inhibiting the absorption of cholesterol (dietary and endogenously-produced), primarily from the small intestine. This inhibition capability is related to the similarity of the plant sterols (stanols) in their physico-chemical properties to that of cholesterol. One generally accepted mechanism by which this inhibition occurs is through competition for space in mixed micelles. Some of the earliest work in this field included studies on the cholesterol-lowering ability of plant sterols in rabbits and in humans (See, Pollak, O. J. (1953a), Reduction of blood cholesterol in man, Circulation 7: 702-706; Pollak, O. J. (1953b), Successful prevention of experimental hypercholesterolemia and cholesterol atherosclerosis in the rabbit, Circulation 7: 696-701, which are herein incorporated by reference in their entireties). Also noteworthy was the fact that in some of these studies rabbits fed plant sterols had significantly reduced atherosclerosis, See Pollak, O. J. (1953b), Successful prevention of experimental hypercholesterolemia and cholesterol atherosclerosis in the rabbit, Circulation 7: 696-701, which is herein incorporated by reference in its entirety, which is the main cause of heart disease in humans. Later work confirmed that plant sterols could significantly reduce serum cholesterol by seventeen percent (17%) in young men with atherosclerotic heart disease, See Farquhar, J. W., Smith, R. E., & Dempsey, M. E. (1956), The effect of β-sitosterol on the serum lipids of young men with arteriosclerotic heart disease, Circulation 14: 77-82, which is herein incorporated by reference in its entirety). Several studies have since reported that the ingestion of plant sterols—or their saturated counterpart, stanols—at levels of 1-4 grams per day is an effective non-drug means of lowering serum LDL cholesterol concentration in humans, See Law, M. (2000), Plant sterol and stanol margarines and health, British Medical Journal 320: 861-864; Nguyen, T. T. (1999), The cholesterol-lowering action of plant stanol esters, Journal of Nutrition 129: 2109-2112, which are herein incorporated by reference in their entireties.

Plant sterols (stanols) are similar in structure to cholesterol but are not made by the human body. Plant sterols (stanols) elicit their cholesterol-lowering effects by blocking the absorption of cholesterol in the small intestine, See Lees, A. M., Mok, H. Y. I., Lees, R. S., McCluskey, M. A., & Grundy, S. M. (1977), Plant sterols as cholesterol-lowering agents: Clinical trials in patients with hypercholesterolemia and studies of sterol balance, Atherosclerosis 28: 325-338; Mattson, F. H., Grundy, S. M., & Crouse, J. R. (1982), Optimizing the effect of plant sterols on cholesterol absorption in man, American Journal of Clinical Nutrition 35: 697-700; Mattson, F. H., Volpenhein, R. A., & Erickson, B. A. (1977), Effect of plant sterol esters on the absorption of dietary cholesterol, Journal of Nutrition 107: 1139-1146, which are herein incorporated by reference in their entireties). In fact, cholesterol absorption is directly correlated with LDL cholesterol concentration, See Gylling, H., & Miettinen, T. A. (1995), The effect of cholesterol absorption inhibition on low density lipoprotein cholesterol level, Atherosclerosis 117: 305-308; Kesaniemi, Y. A., & Miettinen, T. A. (1987), Cholesterol absorption efficiency regulates plasma cholesterol level in the Finnish population, European Journal of Clinical Investigation 17: 391-395; Rudel, L. L., Deckelman, C., Wilson, M. D., Scobey, M., & Anderson, R. (1994), Dietary cholesterol and downregulation of cholesterol 7α-hydroxylase and cholesterol absorption in African green monkeys, Journal of Clinical Investigation 93: 2463-2472, which are herein incorporated by reference in their entireties, such that cholesterol absorption efficiency is now considered a major determinant of serum cholesterol levels in Western populations. Cholesterol absorption is such an important regulator of LDL cholesterol concentration that new drugs are being approved for use that block cholesterol absorption, although long-term mortality and morbidity associated with these drugs is not yet known, See Bays, H. (2002), Ezetimibe, Expert Opinion on Investigational Drugs, 11: 1587-1604; Turley, S. D., & Dietschy, J. M. (2003), The intestinal absorption of biliary and dietary cholesterol as a drug target for lowering the plasma cholesterol level, Preventive Cardiology 6: 29-33, which are herein incorporated by reference in their entireties. Managing cholesterol levels through dietary means is still the most desirable approach. In particular, lowering cholesterol with plant sterols (stanols) is very attractive because, unlike drugs, these natural plant substances are essentially not absorbed in the intestine and, while in the process of blocking cholesterol absorption, are eliminated from the body through normal excretion, See Ostlund, R. E., Jr. (2002), Phytosterols in human nutrition, Annual Review of Nutrition 22: 533-549, which is herein incorporated by reference in its entirety. Plant sterols (stanols) are naturally occurring substances/components found in plants and wood pulp. Plant sterols can be obtained from plant oil sources, such as vegetable oils and tall oil from the wood pulp industry. Various methods of isolation, extraction and recovery of plant sterols have been patented, See U.S. Pat. Nos. 3,993,156; 2,835,682; 2,866,797; 3,691,211; and 4,420,427, which are herein incorporated by reference in their entireties.

It is known that certain fatty acids also naturally lower serum cholesterol concentration by blocking cholesterol absorption. Noticeably lacking in the prior art is a plant sterol (stanol) ester that is combined with fatty acids known to have cholesterol-lowering properties independent of plant sterols. The food industry currently utilizes plant sterols (stanols) that are esterified to fatty acids, forming plant sterol (stanol) esters, derived from vegetable oils or wood tall oil. Unfortunately, the fatty acids being currently utilized in the food industry do not provide independent cholesterol lowering properties. This results in a less than optimum utilization of naturally occurring cholesterol lowering capabilities in a compound containing both the plant substance and fatty acid substance. Therefore it would be desirable to provide a novel plant sterol (stanol) ester compound which utilizes the natural cholesterol lowering properties of both a plant sterol (stanol) and a fatty acid.

Another issue is that currently available plant sterol (stanol) esters, made with vegetable oils, require a significant amount of fat be present in foods (e.g., margarine or salad dressing) in order for the plant sterol (stanol) to be successfully incorporated into the food. Plant sterol (stanol) esters made with vegetable oils yields a soft sticky mass that is not easily dispersed, thus limiting their use to foods high in fat. Therefore, it would be desirable to provide a novel plant sterol (stanol) ester which is capable of being successfully incorporated into various nutritional delivery systems, such as food and food products without requiring significant amounts fat to be present, in order to provide a greater number and more healthful food choices for consumers.

Therefore, it would be desirable to provide compounds with increased cholesterol lowering properties over that of plant sterols (stanols) alone and which may avoid side effects associated with the use of currently available cholesterol lowering drugs. Further, it would be desirable to provide compounds employing fatty acids with increased cholesterol lowering properties over that currently being employed in the food industry. Still further, it would be desirable to provide a compound which allowed for a broader application within a wider variety of nutritional delivery systems (i.e., food and food products) than is currently available. In addition, it would be desirable to provide a method of manufacturing compounds which include the cholesterol lowering properties of both plant sterol (stanol) substances and fatty acids.

### SUMMARY OF THE INVENTION

Accordingly, the present invention provides a novel compound which combines the naturally occurring cholesterol lowering properties of a plant substance, such as plant sterols (stanols), with those of fatty acids, more particularly stearic acid, resulting in a synergistic increase in the lowering of LDL cholesterol. It is an object of the present invention to block cholesterol absorption at the intestinal wall of the small intestine. The cholesterol lowering properties of the present invention may manifest in various mechanisms as contemplated by those of ordinary skill in the art. For example, the novel compound of the present invention may compete for binding sites with cholesterol at the intestinal wall (competition with mixed micelles) and/or interfere with micelle formation.

It is a further object of the present invention to lower cholesterol absorption while assisting in avoiding toxicity which may occur through the use of currently available drug therapies. The novel compound of the present invention acts entirely within the gastrointestinal tract, particularly within the small intestine, and is excreted through the digestive system. The plant sterol(s) (stanol(s)) and fatty acid(s) combination of the present invention avoids being absorbed into various other systems, such as the vascular system, thereby, avoiding toxicity concerns associated with many currently available cholesterol lowering drug therapies.

The present invention allows for the natural cholesterol lowering properties found in the plant sterol (stanol) and fatty acids to be combined in a novel compound, the compound being capable of existing in various states, such as a solid or liquid. The delivery of the novel compound of the present invention may occur via ingestion of the solid or liquid form of the compound, which may increase the effectiveness of receipt of this compound into the gastrointestinal tract of the digestive system of an animal, including mammals, such as human beings, and may assist in lowering serum cholesterol in humans.

It is still another object of the present invention to provide for a cholesterol-lowering compound having enhanced physical characteristics, such as greater solubility and dispersion characteristics (dispersibility), and a wider range of applicability into various nutritional delivery systems, such as food and food products, than other cholesterol lowering products currently available. Increased solubility and dispersibility may result in a higher concentration of the active substances of the cholesterol reducing compound of the present invention reaching the sites where cholesterol absorption may occur and assisting in inhibiting the absorption of the cholesterol.

It is to be understood that both the foregoing general description and the following detailed description are exemplary and explanatory only and are not restrictive of the invention as claimed. The accompanying drawings, which are incorporated in and constitute a part of the specification, illustrate an embodiment of the invention and together with the general description, serve to explain the principles of the invention.

### BRIEF DESCRIPTION OF THE DRAWINGS

The numerous advantages of the present invention may be better understood by those skilled in the art by reference to the accompanying figures in which:

FIG. 1 is an illustration of a stearic acid molecule, a plant sterol molecule, and the resultant product of their esterification, a plant sterol ester;

FIG. 2 is a table illustrating the plasma and liver cholesterol in hamsters fed plant sterol esters;

FIG. 3 is a table illustrating the daily cholesterol output and input in hamsters fed plant sterol esters;

FIG. 4 is a table illustrating the daily cholesterol absorption and excretion in hamsters fed plant sterol esters;

FIG. 5 is a block diagram illustrating a method of receiving a novel compound of the present invention by an animal;

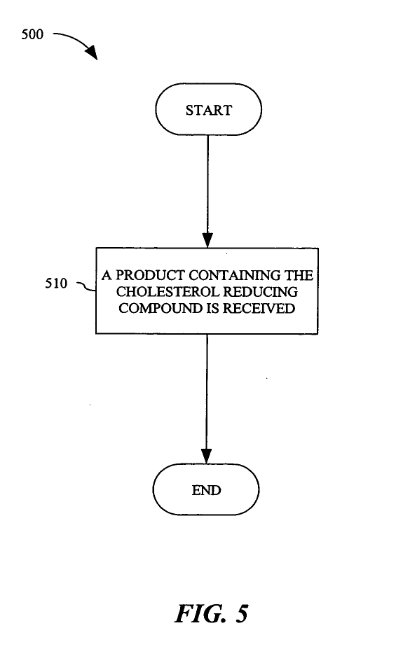

FIG. 6 is a block diagram illustrating a method of manufacturing a novel compound in accordance with an exemplary embodiment of the present invention; and

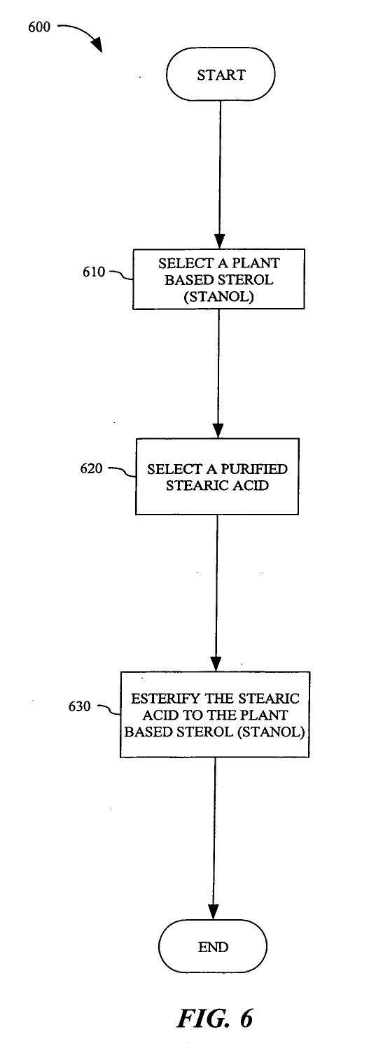

FIG. 7 is a block diagram illustrating a second exemplary method of manufacturing a novel compound of the present invention.

### DETAILED DESCRIPTION OF THE INVENTION

Reference will now be made in detail to the presently preferred embodiments of the invention, examples of which are illustrated in the accompanying drawings.

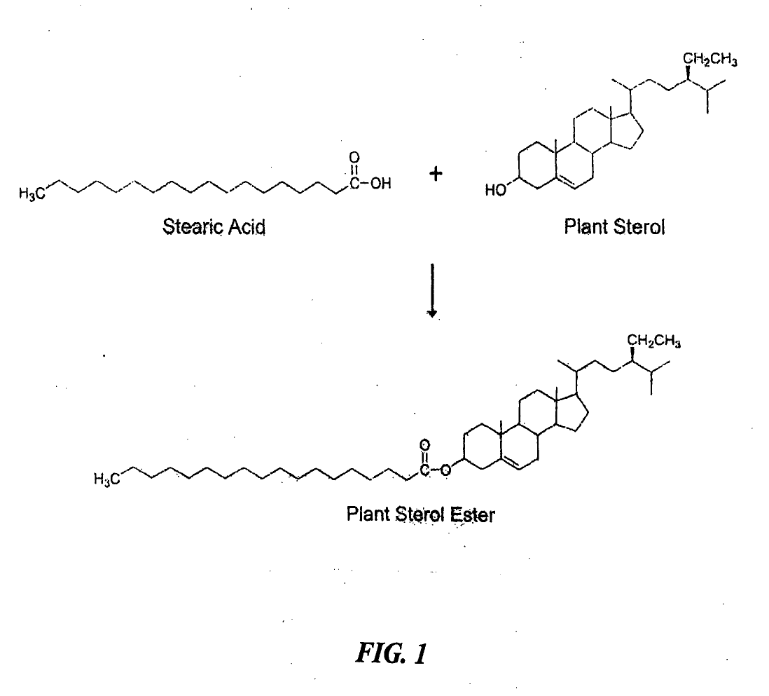

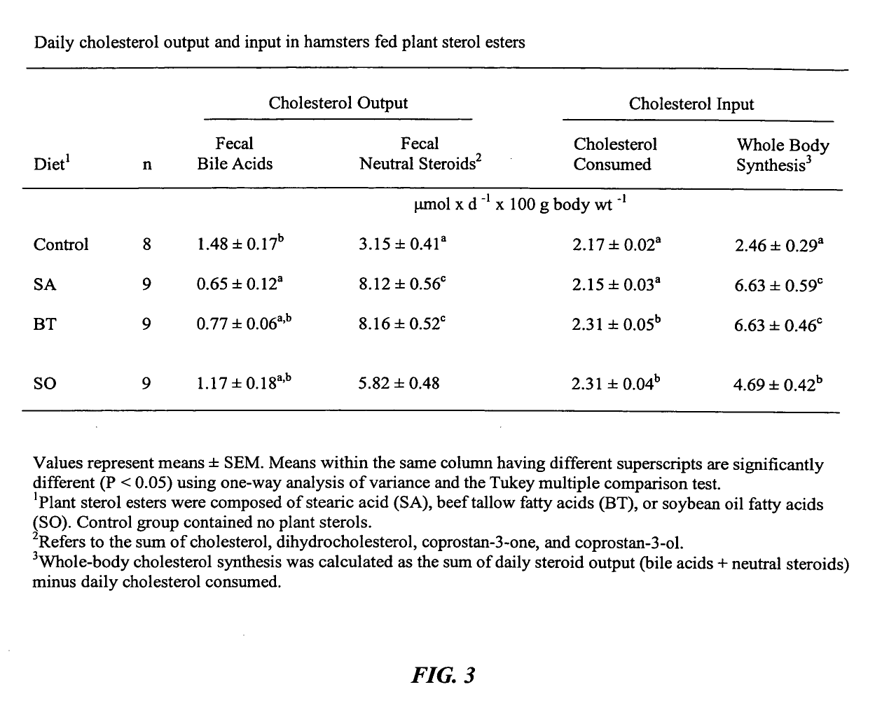

Referring generally now to FIGS. 1 through 4, exemplary embodiments of the present invention are shown. In a preferred embodiment, the novel cholesterol lowering compound of the present invention is a plant sterol ester, as shown in FIG. 1. The plant sterol ester includes a fatty acid, in particular a stearic acid, esterified to a plant based cholesterol reducing substance, in particular a plant sterol. In the alternative, plant stanols, another plant based cholesterol reducing substance, may be esterified by fatty acids, such as the stearic acid. The plant based cholesterol reducing substance will hereinafter be referred to as a plant sterol (stanol), unless otherwise specifically indicated. In the current embodiment of FIG. 1, the stearic acid utilized for the esterification of the plant sterol is a purified stearic acid. The esterification of the fatty acid (stearic acid) to the plant sterol (stanol) may promote the incorporation of the novel compound of the present invention into various nutritional delivery systems, such as food products for consumption by human beings. Thus, the present invention provides a compound which achieves synergistic cholesterol lowering results in animals, such as mammals including human beings, livestock, companion animals and the like, by utilizing the natural cholesterol lowering properties of both a plant sterol (stanol) and fatty acid (stearic acid).

In alternative embodiments, the plant sterol (stanol) may be esterified by a fatty acid including stearic acid, wherein the fatty acid may exist in various forms, such as a mono-glyceride, di-glyceride, and tri-glyceride. These forms of the fatty acid may be contained within whole fat/oil or blends thereof, which include a mixture of fatty acids, such as stearic acid, myristic acid, palmitic acid, trans fatty acid, cis oleic acid, or linoleic acid. In a preferred embodiment, the whole fat/oil or blends thereof, contain a high proportion (concentration) of stearic acid or are enriched with stearic acid. The use of fatty acids which exist in the mono-glyceride, di-glyceride, and tri-glyceride forms allow for the esterification process to proceed. These forms of the fatty acids may be advantageous for use with the present invention because of their relatively easy incorporation within various nutritional delivery systems, such as various food products (as an ingredient) and/or food supplements.

Typical sources of fatty acids may include various oils and fats, such as beef tallow, cocoa butter, cupu assu kernel oil, dhupa oil, gamboge butter, kokum butter, mango seed oil, sal fat, sequa oil, and sheanut oil. It is contemplated that the various oils and fats may be from either vegetable or animal origin. These sources of fatty acids may provide the fatty acids in mono-glyceride, di-glyceride, tri-glyceride, and phospholipid forms. Further, these sources may naturally contain specific fatty acids, such as stearic acid, for use in the novel compound of the present invention. For instance, in the United States, beef tallow is a very inexpensive source of stearic acid and can be used to esterify plant sterol (stanol) in a cost-efficient process.

In a preferred embodiment, the naturally occurring oils and fats include high proportions (concentrations) or non-negligible amounts of the desired fatty acid, specifically stearic acid. It is to be understood that non-negligible amounts of a fatty acid, such as stearic acid, in the fats and oils may preferably provide a thirty percent (30%) concentration of the fatty acid of interest (stearic acid). It is contemplated that the non-negligible amount of the desired fatty acid may range from twenty percent (20%) to one hundred percent (100%) concentration in the present invention. Alternatively, the non-negligible amount may range from ten percent (10%) to one hundred percent (100%) concentration. A negligible amount of a desired fatty acid, such as stearic acid, from fatty acid sources, such as soybean oil, may contain less than ten percent (<10%) concentration of the desired fatty acid. Alternative concentrations of a fatty acid, such as stearic acid, may be considered negligible or non-negligible as contemplated by those of ordinary skill in the art.

Typical sources for the plant sterol may include plants and wood pulp. For instance, it is known that soybeans are a rich source of plant sterol. Additionally, plant sterol may be obtained from plant oil sources, such as vegetable oils and tall oil. Alternative sources for plant sterol may be utilized by the present invention as contemplated by those of ordinary skill in the art without departing from the scope and spirit of the present invention.

The present invention contemplates that the fats and oils, which do not contain high proportions or concentrations of a desired fatty acid, such as stearic acid, may be hydrogenated to increase the concentration of the desired fatty acid, thereby, allowing for their use by present invention. Thus, the oil and fat sources of the fatty acids, such as vegetable oils (e.g., rapeseed, soybean) and tall oil which have negligible amounts of a desired fatty acid, may be enriched through the hydrogenation process with the desired fatty acids, such as stearic acid. This may be advantageous in that the present invention may utilize a broad array of oils and fats as a source, which may or may not contain desired concentrations of specific fatty acids.

The esterification process shown in FIG. 1 may be any one of the widely utilized and known processes used in the food industry. In particular, an esterification process that is primarily used in the food industry is a base-catalyzed reaction involving free sterols and fatty acid methyl esters derived from edible oils, See U.S. Pat. No. 5,522,045, which is herein incorporated by reference in its entirety. A large excess of fatty acid methyl esters are needed to drive the reaction, which also produces methanol, making the purification to food grade material difficult. Alternatively, free sterols can be heated with vegetable oil fatty acids under vacuum without using fatty acid methyl esters, solvents, or catalysts, See U.S. Pat. No. 6,410,758, which is herein incorporated by reference in its entirety.

Converting sterols to stanols by hydrogenation is also common practice in the food industry, See U.S. Pat. No. 5,244,887, which is herein incorporated by reference in its entirety. An early study in rats suggested that stanols might be slightly more effective than sterols in reducing serum cholesterol, See Sugano, M., Morioka, H., & Ikeda, I. (1977), A comparison of hypocholesterolemic activity of β-sitosterol and β-sitostanol in rats, Journal of Nutrition 107: 2011-2019, which herein incorporated by reference in its entirety. However, more recent studies in humans have indicated that sterols are equally effective, See Miettinen, T. A., & Vanhanen, H. (1994), Dietary sitostanol related to absorption, synthesis and serum level of cholesterol in different apolipoprotein E phenotypes, Atherosclerosis 105: 217-226; Weststrate, J. A., & Meijer, G. W. (1998), Plant sterol-enriched margarines and reduction of plasma total—and LDL-cholesterol concentrations in normocholesterolaemic and mildly hypercholesterolaemic subjects. European Journal of Clinical Nutrition 52: 334-343, which are both herein incorporated by reference in their entireties, or even better, See Jones, P. J. H., Raeini-Sarjaz, M., Ntanios, F. Y., Vanstone, C. A., Feng, J. Y., & Parsons, W. E. (2000), Modulation of plasma lipid levels and cholesterol kinetics by phytosterol versus phytostanol esters, Journal of Lipid Research 41: 697-705, which is herein incorporated by reference in its entirety, than stanols in reducing serum LDL cholesterol concentration. At present, it is generally accepted that plant sterols and stanols are equivalent in their cholesterol-lowering abilities, ingested in either the esterified or free form, See Ostlund, R. E., Jr. (2002), Phytosterols in human nutrition, Annual Review of Nutrition 22: 533-549, which is herein incorporated by reference in its entirety.

Thus, the present invention contemplates ester derivatives of the plant sterol (stanol), which may have increased solubility and dispersibility. In a preferred embodiment, the compound of the present invention may be delivered via ingestion and received within the digestive system. The efficacy of the compound of the present invention within the gastrointestinal tract may be a factor of its solubility and dispersibility. Solubility refers to the amount of the compound that may be dissolved within a liquid solution. Increased solubility may allow for an increased amount of the plant based cholesterol reducing substance to be received by an animal, such as a human being, in a single dosage or intake form (i.e., capsule, tablet, supplement, soft gel). Dispersibility refers to the breaking up and distribution of the compound when it is received, such as ingestion into the gastrointestinal tract. Increased dispersiblity may allow for an increased spreading of the compound within the digestive system which may lead to an increase in the area of the gastrointestinal tract that includes the compound which may lead to an increase in the blocking of the absorption of cholesterol.

The novel compound of the present invention may exist in various states, such as a liquid and/or solid. The capability of forming the compound of the present invention in these various states may be advantageous for the receipt, delivery, or administration of the novel compound to animals, such as mammals (i.e., human beings), livestock, companion animals, and the like which have digestive systems. As a solid, the compound of the present invention including the plant sterol (stanol) and the fatty acid (stearic acid) may be configured for ingestion in various forms, such as a food supplement, a tablet, a granule, a capsule (i.e., soft gel capsule), a powder, and the like which may assist in increasing the ability of the compound to be received, delivered, or administered within the digestive system. For example, the novel compound of the present invention may take the form of an energy bar, or powdery mix which may be blended in with a liquid. For instance, the plant sterol (stanol) esters made with stearic acid derived from beef tallow exists as a dry powder which more easily incorporates into food products than currently available plant sterol (stanol) esters. Thus, the synergistic cholesterol lowering effects of the novel compound of the present invention are capable of being included within a wide variety of nutritional delivery systems.

In a liquid state the novel compound of the present invention may be variously configured in an aqueous solution, organic solution, suspension, and emulsion. For example, as a liquid the compound of the present invention may be more easily included within various beverages or liquid foods. Further, the liquid solution may increase the applicability of the compound within various nutritional delivery systems, such as food products, where the liquid compound is preferred over the solid form. An emulsified and/or suspended form may also be incorporated into various nutritional delivery systems where such forms are preferred.

Thus, the present invention may assist a human being in reducing or lowering their serum LDL cholesterol level through intake into the digestive system of a solid or liquid form of the present invention. Further, the novel compound may be useful in therapeutic markets, for example targeting patients with high cholesterol, which may assist in reducing the onset of conditions such as atherosclerosis, and the like. Additional uses pertaining to the treatment of cholesterol and cholesterol related conditions in the livestock/animal food products market and companion animal markets are also within the scope and spirit of the present invention.

Alternatively, it is contemplated that the novel compound of the present invention may be composed of a mixture including a free plant sterol (stanol) and a free fatty acid (stearic acid) form. In such a mixture the stearic acid is provided as an agent of a fatty acid which exists in the form of a mono-glyceride, di-glyceride, tri-glyceride, or phospholipid. In a preferred embodiment, the fatty acid contains a high proportion of stearic acid. Alternatively, the fatty acid being employed may contain only negligible amounts of stearic acid but may be enriched with stearic acid. The mixture form of the novel compound of the present invention may be advantageous over the esterified form in that it may reduce the amount of processing needed to produce the compound.

It is contemplated that both the esterified form and the mixture form of the novel compound of the present invention provides both the plant sterol (stanol) and the fatty acid (stearic acid) in a form which is capable of providing the advantageous cholesterol lowering properties of the two substances. As separate component substances the plant sterol (stanol) and the fatty acid may both promote the blocking of cholesterol absorption. As previously stated, the plant sterol (stanol) may promote the blocking of cholesterol absorption by competing for binding sites with cholesterol at the intestinal wall (competition with mixed micelles) and the fatty acid may interfere with micelle formation. It is contemplated that the mechanism of cholesterol absorption blocking promoted by each substance may vary without departing from the scope and spirit of the present invention.

In the esterified form, the plant sterol (stanol) ester functions to block cholesterol absorption. Masking the carboxylic functional group of the fatty acid (stearic acid) through esterification to the plant sterol (stanol) may result in an increase in delivery efficiency of the plant sterol (stanol) and stearic acid, or other fatty acids which include naturally occurring cholesterol lowering properties, to the intestine where the cholesterol blocking function of the substances takes place. The increased stability and improved solubility and dispersibility of the plant sterol (stanol) and the stearic acid may result in higher concentrations of the component substances in the digestive system, particularly in the intestinal tract, than that which may be achieved through delivery of these components in a non-esterified form. Thus, the lowering of cholesterol concentrations, in particular serum LDL cholesterol concentrations, through the use of the present invention may be increased through the esterification of the plant sterol (stanol) by the stearic acid.

The esterified form of the compound may be broken down into its individual component substances by enzymes, such as esterases, present in the digestive system which may convert the plant sterol (stanol) and fatty acid (stearic acid) back into their respective free forms. The free form of both substances being functionally capable of promoting the blocking of cholesterol absorption, as previously described. It is contemplated that a majority of the esterified form will be cleaved into the individual components inside the gastrointestinal tract via the esterase interaction. Alternatively, various other enzymes, such as gastric lipase, pancreatic lipase, and the like, may cleave off the fatty acids when the fatty acids are in the form of a mono-glyceride, di-glyceride, tri-glyceride, or phospholipid, resulting in free fatty acids and free plant sterol (stanol).

In the esterified form, the compound of the present invention may exhibit advantageous characteristics. In a preferred embodiment, the compound of the present invention may be delivered via ingestion (oral). The esterified compound promotes this ingestible form which may allow for the delivery of more effective amounts of the plant sterol (stanol) and fatty acid of the compound, which may determine its efficacy once introduced into the digestive system. For example, the administration of the plant based cholesterol reducing substance alone may be limited in amount due to the natural biologically processing experienced once received within the digestive system. The esterified form may allow for an increased amount of the plant based cholesterol reducing substance to be received by an animal, such as a human being. The receipt of increased amounts of the esterified plant sterol (stanol) may occur through ingestion of a single intake form (i.e., capsule, tablet, supplement) or through multiple intakes. Accordingly, the present invention has an additional advantage, in that it not only provides a compound having increased cholesterol lowering properties than other products currently available, but it also provides for a compound with increased solubility and other physical characteristics that enhance the ability to incorporate the compound into a wider range of food products.

The present invention involves an esterification process which combines plant based cholesterol reducing substances, such as plant sterols (stanols), with fatty acids, in particular purified stearic acid or a fat/oil including a high proportion of stearic acid or enriched with stearic acid. Noticeably lacking in the prior art is a plant sterol (stanol) ester that is combined with fatty acids known to have cholesterol-lowering properties independent of plant sterols. The food industry currently utilizes plant sterols (stanols) that are esterified to fatty acids derived from vegetable oils (e.g., rapeseed, soybean) or wood tall oil, which do not contain high concentrations of stearic acid. It is contemplated that the novel plant sterol (stanol) esters may be used as a food ingredient, dietary supplement, or incorporated into various nutritional delivery systems in order to assist in lowering serum cholesterol. As shown in FIG. 1, esterifying a typical plant sterol (stanol) with stearic acid results in the novel plant sterol (stanol) ester of the present invention which provides a synergistic cholesterol absorption blocking effect. Thus, the incorporation of plant sterol (stanol) esters such as this in a dietary regimen may provide an increased ability to lower serum cholesterol levels than either the plant sterol (stanol) or the fatty acid (stearic acid) may have independent of the other.

When plant sterols (stanols) are esterified with purified stearic acid or a fat/oil including a high proportion (concentration) of stearic acid or enriched with stearic acid, cholesterol absorption is blocked to a significantly greater extent than when either the plant based cholesterol reducing substance or the stearic acid is consumed individually or when an equivalent amount of plant sterols (stanols) esterified with alternative fatty acids, such as soybean fatty acids, which may contain negligible amounts of stearic acid, are consumed. This synergistic effect provides a significant advantage to the novel compound of the present invention over the currently employed plant sterol (stanol) esters which typically provide only the cholesterol lowering properties of the plant sterol (stanol). Thus, plant sterols (stanols) esterified with stearic acid are highly potent natural substances that, when consumed, may significantly lower serum cholesterol. With the wide range of applicable products and nutritional delivery systems the plant sterol (stanol) ester of the present invention may be incorporated into, the present invention provides a natural, non-drug approach to lowering serum cholesterol and the risk of heart disease and stroke.

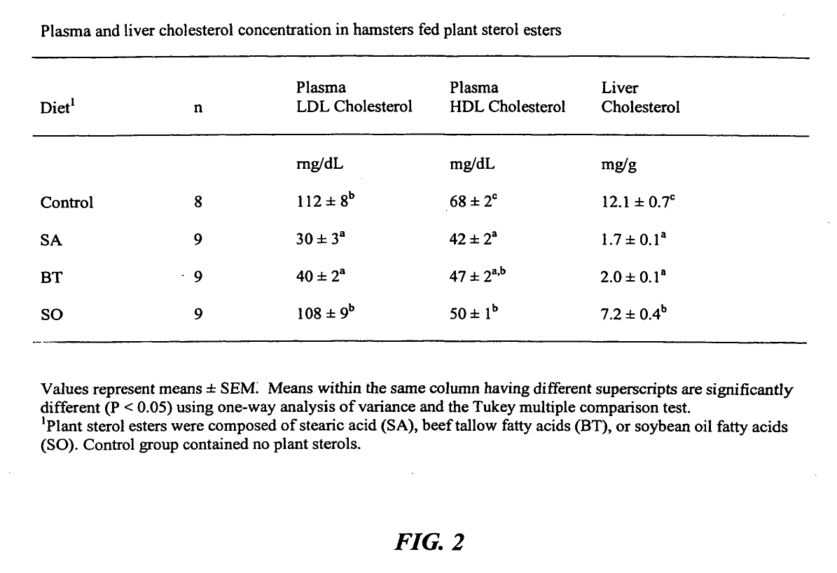

As shown by the data contained in FIG. 2, a group of hamsters fed plant sterol esters, which are the compounds created by the esterification of fatty acids, such as purified stearic acid and/or whole fat/oil or blends thereof, such as beef tallow (high proportion of stearic acid) or oils enriched with non-negligible amounts of stearic acid to plant sterols, had significantly reduced plasma total and LDL cholesterol levels compared to hamsters fed plant sterol esters made with alternative fatty acids, such as soybean oil fatty acids which contains negligible amounts of stearic acid.

The use of various fatty acids in combination with plant sterols has been compared and in the case of stearic acid, which was derived from beef tallow, the combination with a plant sterol to form a sterol ester compound of the present invention provided unexpected results when compared against a plant sterol ester formed from a soybean oil fatty acid, containing negligible amounts of stearic acid, in combination with a plant sterol. In particular it was observed that the average LDL cholesterol concentration in hamsters fed plant sterol esters made with soybean oil fatty acids was 108 mg/dL, whereas the LDL cholesterol concentration in hamsters fed plant sterol esters made with purified stearic acid was only 30 mg/dL. The significance of this is particularly noteworthy because the cholesterol lowering margarines currently available to consumers contain plant sterol (stanol) esters made with vegetable oils that have negligible amounts of stearic acid. Thus, the present invention may be incorporated into known food products and provide an increased reduction in LDL cholesterol concentration in consumers of these food products.

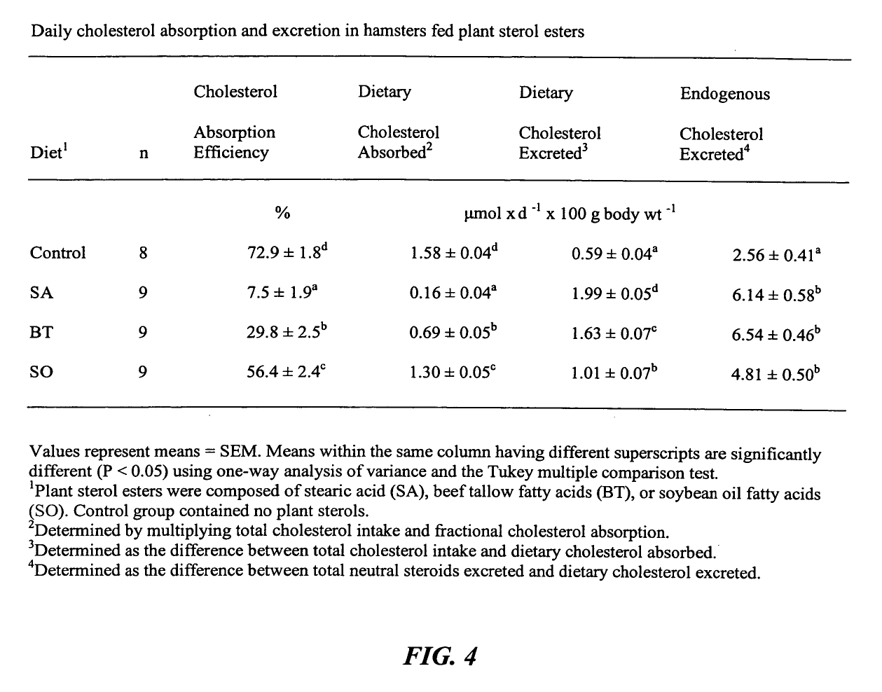

Similar reductions in intestinal cholesterol absorption were observed in hamsters fed plant sterol esters made with purified stearic acid or beef tallow fatty acids, as shown in FIG. 4. Parallel reductions in liver cholesterol was also observed in hamsters fed plant sterol esters made with purified stearic acid and whole fat blends such as beef tallow fatty acids (which contain stearic acid), as shown in FIG. 2. Taken together, the data indicates that: (i) plant sterol esters can be successfully incorporated into ground beef-containing diets, (ii) consumption of ground beef enriched with plant sterol esters significantly lowers both plasma and liver cholesterol concentration, (iii) plant sterol-induced reductions in plasma and liver cholesterol is due to reduced cholesterol absorption, and (iv) plant sterol esters made with beef tallow fatty acids reduce plasma cholesterol, liver cholesterol, and cholesterol absorption to a significantly greater extent than plant sterol esters made with soybean oil fatty acids, as shown in FIGS. 2, 3, and 4.

The present invention, supported by the instant application, may be interpreted to provide beneficial effects in three major areas: (1) Consumers, (2) Food Industry, and (3) Commodities Industry.

First, foods containing plant sterol (stanol) esters made with stearic acid, from sources such as beef tallow, may be an effective cholesterol lowering tool for consumer. Because the cholesterol lowering ability of these plant sterol (stanol) esters is so effective, they may be considered an alternative to drug therapy for cholesterol reduction. This may reduce consumer costs and the side effects experienced by many users of these cholesterol lowering drugs (prescription or non-prescription). As stated previously, the broad application of plant sterol (stanol) esters made with fatty acids, such as stearic acid from sources such as beef tallow, may provide consumers a greater number of food choices. Providing healthier food choices for consumers is important for those looking to optimize their diets while maintaining a busy lifestyle.

For the food industry the sales of nutraceuticals and functional foods, fortified foods, and other “healthy” foods have seen rapid growth in the recent past. For example in 2000, sales of nutraceuticals and these types of foods exceeded $50 billion United States Dollars. Developing food products that fall into these categories may be greatly enhanced because of the broader application of the novel plant sterol (stanol) esters of the present invention produced from the use of fatty acids, such as stearic acid. Another significant factor is the public perception of these food categories. Currently, public perception is very positive which may result in increased demand for these categories of foods. The present invention provides a novel compound which is able to be adapted for use in a wide variety of these food categories in order to assist in meeting consumer demand.

The third benefit may be to the Commodities Industry and particularly to the producers of beef and soybeans. Combining plant sterols (stanols) with beef tallow fatty acids, which includes non-negligible amounts of stearic acid, demonstrates that the present invention provides a useful and “healthful” application for beef tallow which has not been identified or utilized before, and may thus greatly increase the value of tallow surplus which exists. It is further contemplated that the soybean industry may benefit because significant quantities of plant sterol may be produced from soybeans, peanuts, or other naturally occurring sources. Further, the isolation of plant sterols from the soybean, for example, does not disrupt the processing and application of other soy products, such as soy protein. Therefore, increasing the use of beef tallow and soybeans, for the production of fatty acids and plant sterols, respectively, may add value to a currently underutilized component of these and other related commodities.

The present invention provides a method of reducing cholesterol 500. In a first step 510 a product containing the cholesterol lowering compound of the present invention is received by an animal. The product may be incorporated into various nutritional delivery systems, such as food products which incorporate the compound of the present invention as an ingredient or as a food supplement which contains the novel compound. Further, the product may be incorporated into products designed for ingestion which may promote the delivery of the novel compound into the gastrointestinal tract of the digestive system.

In alternative embodiments, a product containing the compound of the present invention may be first selected by user. After the selection the user may receive the compound through any of the above identified applications. Further, in an additional step the compound of the present invention may be incorporated into a dietary regimen. The dietary regimen being designed to provide a cholesterol reducing diet to the follower of the dietary regimen. Thus, the compound of the present invention may promote a healthier lifestyle for its users and through lowering cholesterol, may improve the circulation system and reduce the risk of heart disease.

In another embodiment of the present invention, a method 600 of manufacturing the compound of the present invention is provided. In a first step 610 a plant sterol (stanol) is selected. It is contemplated that prior to the selection of a plant sterol (stanol) a source may be selected. The source may be vegetable (i.e., soybean), plant (i.e., wood tall), and the like, which provide plant sterol (stanol) substances. In step 620 a purified stearic acid is selected as the fatty acid. The purified stearic acid may be collected in its free form or come from various fatty acid sources, as described previously. For instance, the fatty acid source may provide fatty acids in at least one of a mono-glyceride, di-glyceride, and tri-glyceride form, which may be high in stearic acid concentration or through the hydrogenation process may be enriched with stearic acid. In step 630 the purified stearic acid is esterified to the plant sterol (stanol) to form the compound of the present invention. It is contemplated that the stearic acid selected may be in the form of a mono-glyceride, di-glyceride, or tri-glyceride and that this fatty acid may be esterified to the plant sterol (stanol).

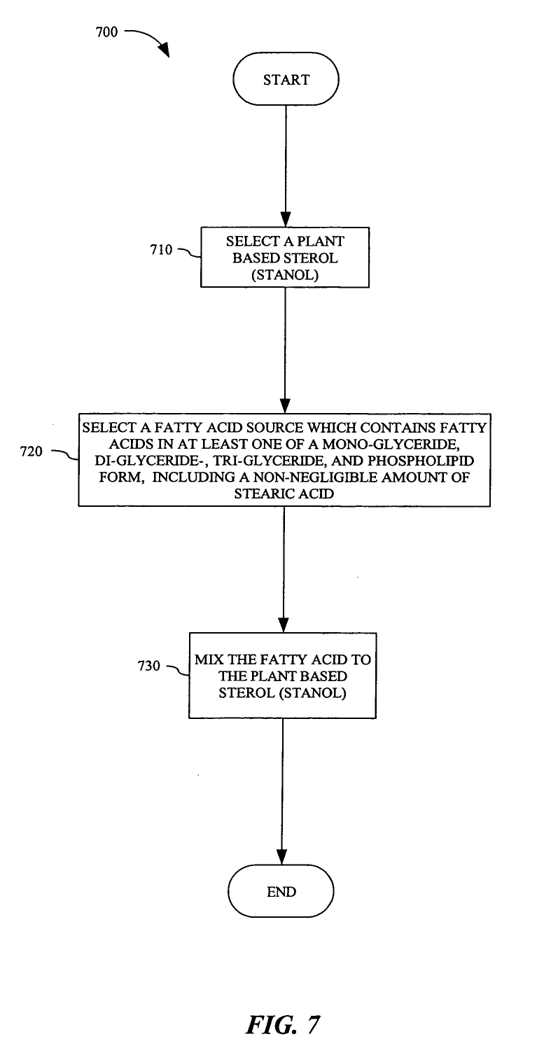

Referring now to FIG. 7, a second exemplary method 700 of manufacturing the compound of the present invention is provided. In a first step 710 a plant sterol (stanol) is selected. As previously described, the plant sterol (stanol) may be derived from various sources, such as vegetables (i.e., soybean), plants (i.e., wood tall), and the like, which are rich sources of plant sterol (stanol). In step 720 a fatty acid source is selected which provides a fatty acid including stearic acid. It is to be understood that the fatty acid source may provide fatty acids in at least one of a mono-glyceride, di-glyceride, tri-glyceride, and phospholipid form, which may be high in stearic acid concentration or through the hydrogenation process may be enriched with stearic acid. In step 730 the fatty acid is mixed with the plant sterol (stanol) to form the compound of the present invention.

It is contemplated that the various methods of manufacture may further include the step of incorporating the compound into a product, such as a food product. The manufacturing method may produce the novel compound in a form which may be utilized as an ingredient in foods or in various nutritional delivery forms. For instance, the manufacturing method may include the step of forming the compound into a food supplement, such as a high energy bar. It is further contemplated that a fatty acid, in any of the various forms previously described, having negligible amounts of stearic acid may be enriched with stearic acid and utilized by the present invention.

It is understood that the specific order or hierarchy of steps in the methods disclosed are examples of exemplary approaches. Based upon design preferences, it is understood that the specific order or hierarchy of steps in the method can be rearranged while remaining within the scope and spirit of the present invention. Further, the source of fatty acids (stearic acid) may vary between the purified stearic acid, the whole fat/oil, or blend thereof, and the enriched oil in the above description without departing from the scope and spirit of the present invention. The accompanying method claims present elements of the various steps in a sample order, and are not necessarily meant to be limited to the specific order or hierarchy presented.

It is believed that the present invention and many of its attendant advantages will be understood by the forgoing description. It is also believed that it will be apparent that various changes may be made in the form, construction and arrangement of the components thereof without departing from the scope and spirit of the invention or without sacrificing all of its material advantages. The form herein before described being merely an explanatory embodiment thereof. It is the intention of the following claims to encompass and include such changes.

1. A cholesterol lowering compound, comprising:
a fatty acid; and
a plant based cholesterol reducing substance selected from the group consisting of a plant sterol or a plant stanol,
wherein the fatty acid is esterified to the plant based cholesterol reducing substance and is suitable for being received by an animal.

2. The compound of claim 1, wherein the fatty acid includes fatty acids selected from the group consisting of a stearic acid, myristic acid, palmitic acid, trans fatty acid, cis oleic acid, and linoleic acid.

3. The compound of claim 2, wherein the fatty acid exists in at least one of a mono-glyceride, di-glyceride, and tri-glyceride form.

4. The compound of claim 3, wherein the mono-glyceride, di-glyceride, and tri-glyceride form contains a high proportion of stearic acid.

5. The compound of claim 3, wherein the mono-glyceride, di-glyceride, and tri-glyceride form is enriched with stearic acid.

6. The compound of claim 1, wherein the fatty acid is derived from a source selected from the group consisting of beef tallow, cocoa butter, cupu assu kernel oil, dhupa oil, gamboge butter, kokum butter, mango seed oil, sal fat, sequa oil, sheanut oil, and hydrogenated oil.

7. The compound of claim 1, wherein the compound is provided in at least one of a liquid and solid state.

8. The compound of claim 7, wherein the solid state of the compound is configured as at least one of a food supplement, a tablet, a granule, a capsule, a soft gel, and a powder.

9. The compound of claim 7, wherein the liquid state of the compound is configured as at least one of an aqueous solution, organic solution, suspension, and emulsion.

10. The compound of claim 7, wherein the compound is received by ingestion.

11. The compound of claim 1, wherein the animal is a mammal selected from the group consisting of at least one of a human, livestock, and companion animals.

12. A cholesterol lowering compound, comprising:
a fatty acid; and
a plant based cholesterol reducing substance selected from the group consisting of a plant sterol or a plant stanol,
wherein the fatty acid is mixed with the plant based cholesterol reducing substance and is suitable for being received by an animal.

13. The compound of claim 12, wherein the fatty acid includes fatty acids selected from the group consisting of a stearic acid, myristic acid, palmitic acid, trans fatty acid, cis oleic acid, and linoleic acid.

14. The compound of claim 13, wherein the fatty acid exists in at least one of a mono-glyceride, di-glyceride, tri-glyceride, and phospholipid form.

15. The compound of claim 14, wherein the mono-glyceride, di-glyceride, tri-glyceride, and phospholipid form contains a high proportion of stearic acid.

16. The compound of claim 14, wherein the mono-glyceride, di-glyceride, tri-glyceride, and phospholipid form is enriched with stearic acid.

17. The compound of claim 12, wherein the fatty acid is derived from a source selected from the group consisting of beef tallow, cocoa butter, cupu assu kernel oil, dhupa oil, gamboge butter, kokum butter, mango seed oil, sal fat, sequa oil, sheanut oil, and hydrogenated oil.

18. The compound of claim 12, wherein the compound is provided in at least one of a liquid and solid state.

19. The compound of claim 18, wherein the solid state of the compound is configured as at least one of a food supplement, a tablet, a granule, a capsule, a soft gel, and a powder.

20. The compound of claim 18, wherein the liquid state of the compound is configured as at least one of an aqueous solution, organic solution, suspension, and emulsion.

21. The compound of claim 18, wherein the compound is received by ingestion.

22. The compound of claim 12, wherein the animal is a mammal selected from the group consisting of at least one of a human, livestock, and companion animals.

23. A cholesterol lowering compound, comprising:
a stearic acid; and
a plant based cholesterol reducing substance selected from a group consisting of a plant sterol and a plant stanol,
wherein the stearic acid is esterified to the plant based cholesterol reducing substance and is suitable for being received by an animal.

24. The compound of claim 23, wherein the stearic acid is derived from a source selected from the group consisting of beef tallow, cocoa butter, cupu assu kernel oil, dhupa oil, gamboge butter, kokum butter, mango seed oil, sal fat, sequa oil, sheanut oil, and hydrogenated oil.

25. The compound of claim 23, wherein the compound is provided in at least one of a liquid and solid state.

26. The compound of claim 25, wherein the solid state of the compound is configured as at least one of a food supplement, a tablet, a granule, a capsule, a soft gel, and a powder.

27. The compound of claim 25, wherein the liquid state of the compound is configured as at least one of an aqueous solution, organic solution, suspension, and emulsion.

28. The compound of claim 25, wherein the compound is received by ingestion.

29. The compound of claim 23, wherein the animal is a mammal selected from the group consisting of at least one of a human, livestock, and companion animals.

30. A cholesterol lowering compound, comprising:
means for providing a fatty acid; and
means for providing a plant based cholesterol reducing substance,
wherein the fatty acid means is esterified to the plant based cholesterol reducing substance means and is suitable for being received by an animal.

31. The compound of claim 30, wherein the means for a fatty acid includes providing a fatty acid selected from the group consisting of a stearic acid, myristic acid, palmitic acid, trans fatty acid, cis oleic acid, and linoleic acid.

32. The compound of claim 31, wherein the fatty acid is a purified stearic acid.

33. The compound of claim 31, wherein the fatty acid exists in at least one of a mono-glyceride, di-glyceride, and tri-glyceride form.

34. The compound of claim 33, wherein the mono-glyceride, di-glyceride, and tri-glyceride form contains a high proportion of stearic acid.

35. The compound of claim 33, wherein the mono-glyceride, di-glyceride, and tri-glyceride form is enriched with stearic acid.

36. The compound of claim 31, wherein the fatty acid is mixed with the plant based cholesterol reducing substance means.

37. The compound of claim 36, wherein the fatty acid exists in at least one of a mono-glyceride, di-glyceride, tri-glyceride, and phospholipid form.

38. The compound of claim 30, wherein the means for providing a plant based cholesterol reducing substance includes providing a plant based cholesterol reducing substance from a group consisting of a plant sterol and a plant stanol.

39. A method for lowering cholesterol in an animal, comprising:
receiving of a cholesterol lowering compound including a fatty acid esterified to a plant based cholesterol reducing substance by an animal.

40. The method of claim 39, wherein the fatty acid includes fatty acids selected from the group consisting of a stearic acid, myristic acid, palmitic acid, trans fatty acid, cis oleic acid, and linoleic acid.

41. The method of claim 40, wherein the fatty acid is a purified stearic acid.

42. The method of claim 40, wherein the fatty acid exists in at least one of a mono-glyceride, di-glyceride, and tri-glyceride form.

43. The method of claim 42, wherein the mono-glyceride, di-glyceride, and tri-glyceride form contains a high proportion of stearic acid.

44. The method of claim 42, wherein the mono-glyceride, di-glyceride, and tri-glyceride form is enriched with stearic acid.

45. The method of claim 40, wherein the fatty acid is mixed with the plant based cholesterol reducing substance.

46. The method of claim 45, wherein the fatty acid exists in at least one of a mono-glyceride, di-glyceride, tri-glyceride, and phospholipid form.

47. The method of claim 39, wherein the plant based cholesterol reducing substance is selected from a group consisting of a plant sterol and a plant stanol.

48. The method of claim 39, further comprising the step of preparing the compound in at least one of a liquid and solid state.

49. The method of claim 48, wherein receiving occurs by ingestion.

50. The method of claim 39, further comprising the step of incorporating the receiving of the compound into a dietary regimen.

51. The method of claim 39, wherein the animal is a mammal selected from the group consisting of at least one of a human, livestock, and companion animal.

52. A method of manufacturing a cholesterol lowering compound, comprising:
selecting a plant based cholesterol reducing substance;
selecting a fatty acid; and
forming a compound including the selected fatty acid and the selected plant based cholesterol reducing substance.

53. The method of claim 52, wherein the plant based cholesterol reducing substance is selected from a plant sterol or a plant stanol.

54. The method of claim 52, wherein the fatty acid is selected from a group consisting of a stearic acid, myristic acid, palmitic acid, trans fatty acid, cis oleic acid, and linoleic acid.

55. The method of claim 54, wherein the step of forming a compound is through esterification of the fatty acid to the plant based cholesterol reducing substance.

56. The method of claim 55, wherein the fatty acid exists in at least one of a purified stearic acid, a mono-glyceride, di-glyceride, and tri-glyceride form.

57. The method of claim 54, wherein the step of forming a compound is through mixing the fatty acid with the plant based cholesterol reducing substance.

58. The method of claim 57, wherein the fatty acid exists in at least one of a mono-glyceride, di-glyceride, tri-glyceride, and phospholipid form.

59. The method of claim 52, further comprising the step of deriving the fatty acid from a source selected from the group consisting of beef tallow, cocoa butter, cupu assu kernel oil, dhupa oil, gamboge butter, kokum butter, mango seed oil, sal fat, sequa oil, sheanut butter, and hydrogenated oil.

60. The method of claim 52, wherein the compound is in at least one of a liquid and solid state.
